(19) United States
(12) Patent Application Publication
(10) Pub. No.: US 2008/0076788 A1
(43) Pub. Date: Mar. 27, 2008

(54) SOLID FORMS OF (3'-CHLOROBIPHENYL-4-YL)(1-(PYRIMIDIN-2-YL)PIPERIDIN-4-YL)METHANONE AND METHODS OF THEIR USE

(76) Inventors: Joseph BARBOSA, Lambertville, NJ (US); Cynthia Anne Fink, Lebanon, NJ (US); Michael Alan Green, Easton, PA (US)

Correspondence Address: LEXICON PHARMACEUTICALS, INC., 8800 TECHNOLOGY FOREST PLACE, THE WOODLANDS, TX, 77381-1160, US

(21) Appl. No.: 11/832,728

(22) Filed: Aug. 2, 2007

Related U.S. Application Data
(60) Provisional application No. 60/835,677, filed on Aug. 4, 2006.

(51) Int. Cl. A61K 31/505; A61P 25/00; C07D 401/04

(52) U.S. Cl. 514/275; 544/324

(57) ABSTRACT

Solid amorphous and crystalline forms of (3′-chlorobiphenyl-4-yl)(1-(pyrimidin-2-yl)piperidin-4-yl)methanone are disclosed, in addition to methods of their use in the treatment of various diseases and disorders.

This application claims priority to U.S. provisional patent application No. 60/835,677, filed Aug. 4, 2006, the entirety of which is incorporated herein by reference.

### 1. FIELD OF THE INVENTION

This invention relates to amorphous and crystalline solid forms of (3′-chlorobiphenyl-4-yl)(1-(pyrimidin-2-yl)piperidin-4-yl)methanone, and to methods of their use.

### 2. BACKGROUND OF THE INVENTION

Different solid forms of the same compound can have substantially different properties. For example, the amorphous form of a drug may exhibit different dissolution characteristics and different bioavailability patterns than its crystalline form(s), properties which can affect how the drug must be administered to achieve optimal effect. Amorphous and crystalline forms of a drug may also have different handling properties (e.g., flowability, compressibility), dissolution rates, solubilities and stabilities, all of which can affect the manufacture of dosage forms. Consequently, access to multiple forms of a drug is desirable for a variety of reasons. Moreover, regulatory authorities (e.g., the U.S. Food and Drug Administration) may require the identification of all solid (e.g., polymorphic) forms of a new drug substance before approving products containing it. A. Goho, Science News 166(8):122-123 (2004).

Compounds may exist in one or more crystalline forms, but their existence and characteristics cannot be predicted with any certainty. In addition, no standard procedure exists for the preparation of all possible polymorphic forms of a compound. And even after one polymorph has been identified, the existence and characteristics of other forms can only be determined by additional experimentation. Id.

### 3. SUMMARY OF THE INVENTION

This invention is directed, in part, to amorphous and crystalline solid forms of (3′-chlorobiphenyl-4-yl)(1-(pyrimidin-2-yl)piperidin-4-yl)methanone, which is an inhibitor of the Na⁺-dependent proline transporter.

One embodiment of the invention encompasses pharmaceutical compositions comprising the solid forms described herein.

Another embodiment encompasses methods of improving cognitive performance, and of treating, managing and/or preventing various diseases and disorders, using solid forms of the invention.

### 4. BRIEF DESCRIPTION OF THE FIGURES

Certain aspects of the invention may be understood with reference to the attached figures.

FIG. 1 is a X-ray diffraction pattern of a crystalline solid form of (3′-chlorobiphenyl-4-yl)(1-(pyrimidin-2-yl)piperidin-4-yl)methanone. The spectrum was obtained using a Shimadzu XRD-6000 diffractometer configured as follows: X-ray tube[Cu (1.54060 Å), 40.0 kV, 40.0 mA]; scan range [3.00 to 45.0 degrees, 0.0400 degree step size]; count time [1.20 sec].

FIG. 2 is a FT-Raman spectrum of a crystalline solid form of (3′-chlorobiphenyl-4-yl)(1-(pyrimidin-2-yl)piperidin-4-yl)methanone. The spectrum was obtained using a Bruker RFS100 spectrometer: 1064 nm excitation (100 mW); 64 scans.

### 5. DETAILED DESCRIPTION OF THE INVENTION

This invention is directed, in part, to solid amorphous and crystalline forms of (3′-chlorobiphenyl-4-yl)(1-(pyrimidin-2-yl)piperidin-4-yl)methanone, which is an inhibitor of the Na⁺-dependent proline transporter. See U.S. patent application Ser. Nos. 11/433,057 and 11/433,626, both filed May 12, 2006. When administered to mice, the compound has been shown to increase learning and memory.

This invention is also directed to dosage forms comprising solid amorphous and crystalline forms of (3′-chlorobiphenyl-4-yl)(1-(pyrimidin-2-yl)piperidin-4-yl)methanone, and to methods of using solid amorphous and crystalline forms of (3′-chlorobiphenyl-4-yl)(1-(pyrimidin-2-yl)piperidin-4-yl)methanone for the improvement of cognitive performance and for the treatment, prevention and/or management of diseases and disorders such as Alzheimer's disease, autism, cognitive disorders, dementia, learning disorders, and short- and long-term memory loss.

### 5.1. Definitions

Unless otherwise indicated, the terms “manage,” “managing” and “management” encompass preventing the recurrence of the specified disease or disorder, or of one or more of its symptoms, in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the terms “prevent,” “preventing” and “prevention” contemplate an action that occurs before a patient begins to suffer from the specified disease or disorder, which inhibits or reduces the severity of the disease or disorder, or of one or more of its symptoms. The terms encompass prophylaxis.

Unless otherwise indicated, a “prophylactically effective amount” of a compound is an amount sufficient to prevent a disease or condition, or one or more symptoms associated with the disease or condition, or to prevent its recurrence. A prophylactically effective amount of a compound is an amount of therapeutic agent, alone or in combination with other agents, which provides a prophylactic benefit in the prevention of the disease or condition. The term “prophylactically effective amount” can encompass an amount that improves overall prophylaxis or enhances the prophylactic efficacy of another prophylactic agent.

Unless otherwise indicated, a “therapeutically effective amount” of a compound is an amount sufficient to provide a therapeutic benefit in the treatment or management of a disease or condition, or to delay or minimize one or more symptoms associated with the disease or condition. A therapeutically effective amount of a compound is an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or one or more of its symptoms, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should also be noted that any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit. Structures that represent compounds with one or more chiral centers, but which do not indicate stereochemistry (e.g., with bolded or dashed lines), encompasses pure stereoisomers and mixtures (e.g., racemic mixtures) thereof. Similarly, names of compounds having one or more chiral centers that do not specify the stereochemistry of those centers encompass pure stereoisomers and mixtures thereof.

### 5.2. Forms of (3′-Chlorobiphenyl-4-yl)(1-(pyrimidin-2-yl)piperidin-4-yl)methanone

This invention is directed to solid forms of (3′-chlorobiphenyl-4-yl)(1-(pyrimidin-2-yl)piperidin-4-yl)methanone, the structure of which is depicted below: 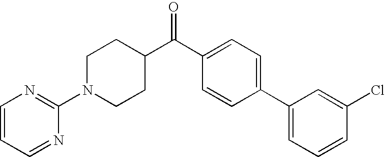
One embodiment is directed to a solid amorphous form. Another is directed to a solid crystalline form.

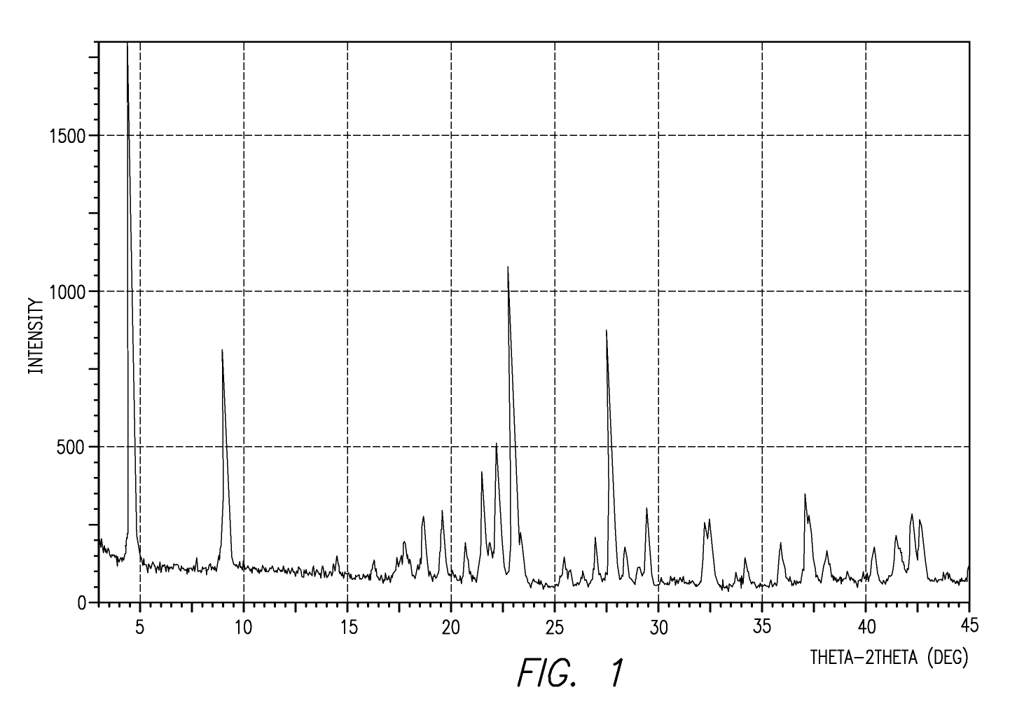

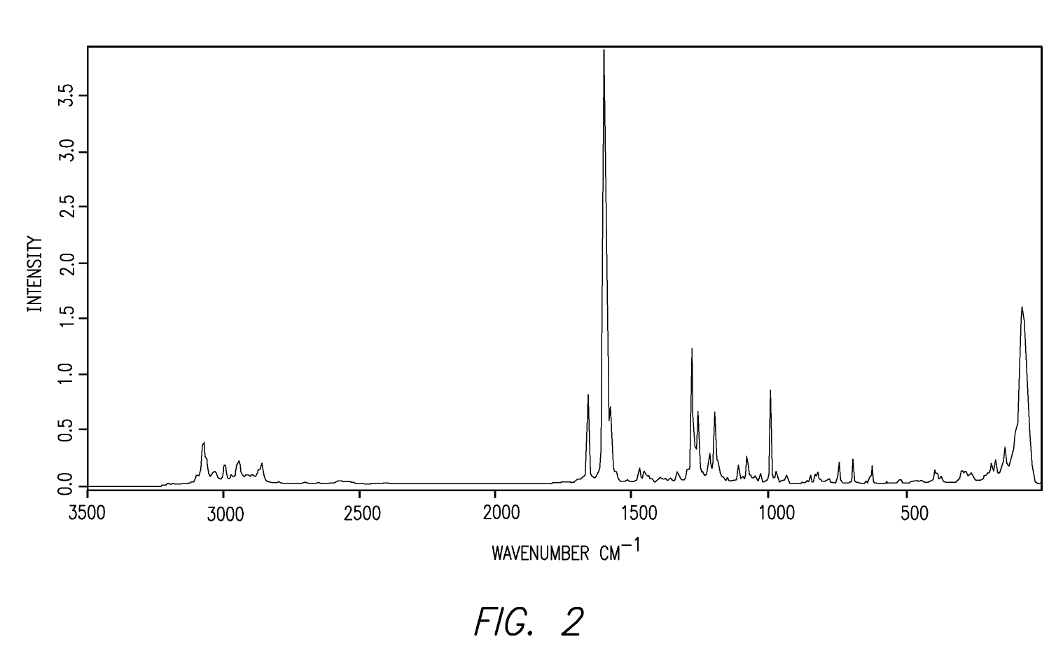

A particular crystalline form of the compound has a melting point of about 117° C. (e.g., ±1.5° C.) as determined by DSC. The form provides a X-ray diffraction pattern that contains peaks at about 4.7, 9.3, 18.8, 19.7, 22.4, 23.2, 27.9, 29.6, 32.3, 32.6, 37.2, 41.5, 42.3, and/or 42.7 degrees 2θ. As those skilled in the art are well aware, the relative intensities of peaks in a X-ray diffraction pattern of a crystalline form can vary depending on how the sample is prepared and how the data is collected. With this in mind, an example of a X-ray diffraction pattern of this crystalline form is provided in FIG. 1. An example of an FT-Raman spectrum of this crystalline form is provided in FIG. 2.

This particular crystalline form can be prepared by: dissolving 3′-chlorobiphenyl-4-yl)(1-(pyrimidin-2-yl)piperidin-4-yl)methanone in ethanol (e.g., at a temperature greater than room temperature) to provide a solution; cooling the solution (or allowing the solution to cool) to a temperature at which crystalline 3′-chlorobiphenyl-4-yl)(1-(pyrimidin-2-yl)piperidin-4-yl)methanone forms; and isolating the crystalline 3′-chlorobiphenyl-4-yl)(1-(pyrimidin-2-yl)piperidin-4-yl)methanone.

This invention encompasses solids that are mixtures of both amorphous and crystalline forms. Certain such solids comprise crystalline (3′-chlorobiphenyl-4-yl)(1-(pyrimidin-2-yl)piperidin-4-yl)methanone in an amount of at least about 50, 75, 80, 85, 90, 95 or 99 weight percent.

### 5.3. Methods of Treatment

One embodiment of this invention encompasses a method of inhibiting a proline transporter, which comprises contacting a proline transporter (in vitro or in vivo) with a sufficient amount of a compound of the invention (i.e. a compound disclosed herein). Preferred proline transporters are encoded by the human gene SLC6A7, the murine ortholog thereof, or a nucleic acid molecule that encodes a proline transporter and that hybridizes under standard conditions to the full length of either.

Another embodiment encompasses a method of improving the cognitive performance of a human patient, which comprises administering to the patient an effective amount of a compound of the invention. Examples of improved cognitive performance include enhanced learning (e.g., learning more quickly), improved comprehension, improved reasoning, and improved short- and/or long-term memory.

Another embodiment encompasses a method of treating, managing or preventing a cognitive disorder (e.g., difficulty in thinking, reasoning, or problem solving), memory loss (short- and long-term), or a learning disorder (e.g., dyslexia, dyscalculia, dysgraphia, dysphasia, dysnomia), which comprises administering to the patient an effective amount of a compound of the invention.

Another embodiment encompasses a method of treating, managing or preventing a disease or disorder, or a cognitive impairment associated therewith, in a human patient, which comprises administering to the patient a therapeutically or prophylactically effective amount of a compound of the invention. Examples of diseases and disorders include age-associated memory impairment, Alzheimer's disease, Attention-Deficit/Hyperactivity Disorder (ADD/ADHD), autism, Down syndrome, Fragile X syndrome, Huntington's disease, Parkinson's disease, and schizophrenia. Additional disorders include adverse sequelae of brain damage caused by, for example, oxygen starvation, traumatic injury, heart attack or stroke.

The invention also encompasses methods of treating, preventing and managing dementia, including dementia associated with metabolic-toxic, structural and/or infectious causes.

Metabolic-toxic causes of dementia include: anoxia; B₁₂ deficiency; chronic drug, alcohol or nutritional abuse; folic acid deficiency; hypercalcemia associated with hyperparathyroidism; hypoglycemia; hypothyroidism; organ system failure (e.g., hepatic, respiratory, or uremic encephalopathy); and pellagra.

Structural causes of dementia include: amyotrophic lateral sclerosis; brain trauma (e.g., chronic subdural hematoma, dementia pugilistica); brain tumors; cerebellar degeneration; communicating hydrocephalus; irradiation to frontal lobes; multiple sclerosis; normal-pressure hydrocephalus; Pick's disease; progressive multifocal leukoencephalopathy; progressive supranuclear palsy; surgery; vascular disease (e.g., multi-infarct dementia); and Wilson's disease.

Infectious causes of dementia include: bacterial endocarditis; Creutzfeldt-Jakob disease; Gerstmann-Sträussler-Scheinker disease; HIV-related disorders; neurosyphilis; tuberculous and fungal meningitis; and viral encephalitis.

The amount of solid form administered to a patient will depend on the route of administration and on the condition being treated, managed or prevented, and can be readily determined by physicians. Example dosing regimens include: 150, 600 and 1200 mg/day by oral administration.

### 5.4. Pharmaceutical Compositions

This invention encompasses pharmaceutical compositions and dosage forms comprising solid form of the invention. Pharmaceutical compositions and dosage forms of this invention may optionally contain one or more pharmaceutically acceptable carriers or excipients. Certain pharmaceutical compositions are single unit dosage forms suitable for oral, topical, mucosal (e.g., nasal, pulmonary, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The formulation should suit the mode of administration. For example, oral administration may require enteric coatings to protect the active ingredient from degradation within the gastrointestinal tract. In another example, the active ingredient may be administered in a liposomal formulation to shield it from degradative enzymes, facilitate transport in circulatory system, and/or effect delivery across cell membranes to intracellular sites.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

### 6. EXAMPLES

The compound (3′-chloro-biphenyl-4-yl)-(1-pyrimidin-2-yl-piperidin-4-yl)-methanone was prepared from an intermediate, (3′-chloro-biphenyl-4-yl)-piperidine-4-yl-methanone hydrochloride, as described below.

### 6.1. Preparation of (3′-Chloro-biphenyl-4-yl)-piperidine-4-yl-methanone hydrochloride

(3′-Chloro-biphenyl-4-yl)-piperidine-4-yl-methanone hydrochloride was prepared by three different methods, identified below as A, B and C.

Method A:

3-Chlorophenyl boronic acid (Alfa Aesar, purity 97%)(40.7 g, 261.19 mmol, 1.4 eq) was dissolved in isopropanol (Aldrich, ACS reagent grade) (800 ml) under nitrogen atmosphere. This was added to a solution of aqueous potassium carbonate (77 g in 150 ml water), bis(triphenylphosphine)palladium(II) dichloride (PdCl₂(PPh₃)₂) (0.65 g, 0.93 mmol, 0.5 mol. eq.) and (4-bromophenyl)(piperidine-4-yl)methanone (50 g, 187 mmol, 1 eq) were stirred at 80° C. for three hours and deemed complete by LC/MS. After the reaction mixture cooled down to 50° C., it was filtered through celite pad, washed with methanol (1 liter). The filtrate was diluted with water (200 ml), then the organic solvent removed under reduced pressure. The resulting crude product was dissolved in ethyl acetate (800 ml) and washed with 1N sodium hydroxide (2×40 ml) and water (1×40 ml).

The organic layer was stirred with aqueous lactic acid (64 g of 85% lactic acid in 600 ml of water) at 50° C. for 20 minutes. After the organic layer was separated (solution assay indicated 8% of product present in the organic layer, which can be captured by additional lactic acid extraction), the aqueous layer was washed with ethyl acetate (2×100 ml). The aqueous layer was separated, basified to pH=11 with 25% NaOH (˜70 ml), and then extracted with ethyl acetate (2×200 ml), dried over sodium sulfate, filtered and concentrated under reduced pressure to obtained biaryl product 46.23 g (83%) as a syrup. HPLC indicated 99.4% product and 0.57% of debrominated staring material.

The above product was dissolved in mixture of ethyl acetate (900 ml) and ethanol (45 ml) and heated at 50° C. 6M aq. HCl (40 ml) was added dropwise over a period of ten minutes. After 20 minutes, the reaction mixture was cooled to room temperature, and stirring was continued for an additional hour. The resulting white solid was filtered and dried under vacuum at 50° C. for five hours to afford 49.8 grams of the biaryl HCl salt (80%). HPLC indicated pure product.

¹H NMR (DMSO-d₆) δ: 1.92(m,4H), 2.52(m,2H), 3.12(m, 2H), 3.82(m,1H), 7.51(m,2H),7.75 (m,1H), 7.82(br s,1H), 7.92(bs d, 2H), 8.12(brd, 2H), 9.0 (br s, 2H). MH⁺=300, 302 (about 3:1). Pd: 15 ppm.

Method B

A round bottom flask was charged with (4-bromophenyl)(piperidine-4-yl)methanone (20.0 g, 74.6 mmol), 3-chlorophenyl boronic acid (17.4 g, 111 mmol, 1.5 eq), and palladium encapsulated catalyst (Aldrich, Pd EnCat-TPP®, catalyst species PdCl₂(PPh₃)₂) (5.2 g, 0.187 mmol, 0.05 eq). These solids were suspended in isopropanol (570 ml) and allowed to stir for five minutes. To the mixture was added potassium carbonate (30.8 g, 224 mmol, 3 eq) dissolved in H₂O (30 ml). The reaction mixture was heated to 80° C. for 16 hours and deemed complete by LC/MS. The suspension was filtered through a small bed of Celite® and the filtrate was concentrated to dryness. The resulting solids were dissolved in isopropyl acetate (400 ml) and washed with water (3×75 ml). The organic layer was then cooled to 0° C. (ice/water bath) and to this stirring solution was added slowly 6 N HCl until solids crystalized. The solids were filtered and dried in a vacuum oven for 16 hours at 50° C. to afford 16.9 g of compound (68% yield) in >98% HPLC purity. MH⁻=300, 302 (about 3:1). Pd=3 ppm.

Method C

A round bottom flask was charged with (4-bromophenyl)(piperidine-4-yl)methanone (4.00 g, 14.9 mmol), 3-chlorophenyl boronic acid (3.26 g, 20.9 mmol, 1.4 eq), and Fibrecat 1029® (0.70 g, 0.448 mmol, 0.03 eq, Johnson Matthey). These solids were suspended in isopropanol (68 ml) and allowed to stir for five minutes. To this stirring solution was added potassium carbonate (6.18 g, 44.8 mmol, 3 eq.) dissolved in H₂O (12 ml). The resulting solution was heated to 80° C. for 16 hours, at which time the reaction was deemed complete by LC/MS. The reaction mixture was filtered through a small bed of Celite® and the filtrate was concentrated to dryness. The resulting solids were dissolved in isopropyl acetate (100 ml) and washed with water (3×50 ml). The organics were cooled to 0° C., and to this stirring mixture was added slowly 6N HCl until solids crashed out of solution. The solids were filtered and dried for 16 hours at 50° C. in a vacuum oven to afford 2.89 g of compound (72%) in >98% HPLC purity. MH⁺=300, 302 (about 3:1). Pd: 4 ppm

### 6.2. Preparation of (3′-Chloro-biphenyl-4-yl)-(1-pyrimidin-2yl-piperidine-4-yl)-methanone

A mixture of (3′-chloro-biphenyl-4-yl)-piperidine-4-yl-methanone hydrochloride salt (40 g, 119.4 mmol), 2-chloropyrimidine (19 g, 167.16 mmol, 1.4 eq), potassium carbonate (325 mesh, Aldrich) (49.4 g, 358.2 mmol, 3 eq) and acetonitrile (560 ml) were stirred at 60° C. for 14 hours and deemed complete by LC/MS. The reaction mixture was concentrated, and the residue was dissolved in ethyl acetate (800 ml) and water (200 ml). The aqueous layer was separated and extracted with ethyl acetate (1×200 ml). The organic layers were combined and washed with brine (1×50 ml), dried and concentrated.

### 6.3. Crystallization of (3′-Chloro-biphenyl-4-yl)-(1-pyrimidin-2yl-piperidine-4-yl)-methanone

The product obtained as described in Example 2 was taken into ethanol (700 ml) and stirred with a mechanical stirrer at 70° C. After 30 minutes, the solid was completely dissolved. At that time, the reaction temperature was decreased to 45° C. and stirred for 4 hours (heterogeneous mixture). The reaction mixture was then stirred at room temperature for 3 hours. The resulting white solid was filtered washed with ethanol (50 ml), dried at 50° C. for 5 hours. This gave the product in 84.4% yield (33.9 g) as a white solid. 100% pure by HPLC.

¹H NMR(CDCl₃) δ: 1.75(m,2H), 1.92(m,2H), 3.05(m,2H), 3.50(m,1H), 4.75(m,2H), 6.42(t,1H), 7.32(m,2H), 7.4(m,1H), 7.51(s,1H),7.62(d,2H), 7.95(d,2H), 8.22(d,2H). ¹³CNMR(CDCl₃): 28.72, 43.79,44.34, 110.2, 125.82, 127.78, 127.81, 128.63, 129.39, 130.62, 135.29, 135.49, 142.03, 144.62, 158.13, 161.93, 202.09. MH⁺=378, 380 (about 3:1 ratio). Pd: 2 ppm.

All patents and patent applications cited above are incorporated herein by reference in their entireties. The full scope of the invention is understood with reference to the appended claims.

1. Solid amorphous (3′-chlorobiphenyl-4-yl)(1-(pyrimidin-2-yl)piperidin-4-yl)methanone or a pharmaceutically acceptable salt or solvate thereof.

2-9. (canceled)

10. A crystalline compound, wherein the compound is (3′-chlorobiphenyl-4-yl)(1-(pyrimidin-2-yl)piperidin-4-yl)methanone or a pharmaceutically acceptable salt or solvate thereof.

11. The crystalline compound of claim 10, which has an X-ray powder diffraction pattern that comprises a peak at about 4.7 degrees 2θ.

12. The crystalline compound of claim 10, which has an X-ray powder diffraction pattern that comprises peaks at about 9.3 and 18.8 degrees 2θ.

13. The crystalline compound of claim 10, which has an X-ray powder diffraction pattern that comprises peaks at about 19.7 and 22.4 degrees 2θ.

14. The crystalline compound of claim 10, which has an X-ray powder diffraction pattern that comprises peaks at about 23.2 and 27.9 degrees 2θ.

15. The crystalline compound of claim 10, which has an X-ray powder diffraction pattern that comprises peaks at about 29.6 and 32.2 degrees 2θ.

16. The crystalline compound of claim 10, which has an X-ray powder diffraction pattern that comprises peaks at about 32.6 and 37.2 degrees 2θ.

17. The crystalline compound of claim 10, which has an X-ray powder diffraction pattern that comprises peaks at about 41.6 and 42.3 degrees 2θ.

18. The crystalline compound of claim 10, which has an X-ray powder diffraction pattern that comprises peaks at about 9.3, 27.9 and 42.7 degrees 2θ.

19. The crystalline compound of claim 10, which has an X-ray powder diffraction pattern that is substantially the same as that shown in FIG. 1.

20. The crystalline compound of claim 10, which has a Raman spectrum that is substantially the same as that shown in FIG. 2.

21. The crystalline compound of claim 10, which has a melting point of about 117° C.

22. Solid 3′-chlorobiphenyl-4-yl)(1-(pyrimidin-2-yl)piperidin-4-yl)methanone comprising crystalline 3′-chlorobiphenyl-4-yl)(1-(pyrimidin-2-yl )piperidin-4-yl)methanone in an amount of at least about 50 weight percent.

23. The solid of claim 22, which comprises crystalline 3′-chlorobiphenyl-4-yl)(1-(pyrimidin-2-yl)piperidin-4-yl)methanone in an amount of at least about 75 weight percent.

24. The solid of claim 23, which comprises crystalline 3′-chlorobiphenyl-4-yl)(1-(pyrimidin-2-yl)piperidin-4-yl)methanone in an amount of at least about 95 weight percent.

25. A method of preparing crystalline 3′-chlorobiphenyl-4-yl)(1-(pyrimidin-2-yl)piperidin-4-yl)methanone, which comprises:
dissolving 3′-chlorobiphenyl-4-yl)(1-(pyrimidin-2-yl)piperidin-4-yl)methanone in ethanol to provide a solution;
cooling the solution to a temperature at which crystalline 3′-chlorobiphenyl-4-yl)(1-(pyrimidin-2-yl)piperidin-4-yl)methanone forms; and
isolating the crystalline 3′-chlorobiphenyl-4-yl)(1-(pyrimidin-2-yl)piperidin-4-yl)methanone.

26. A pharmaceutical dosage form comprising the crystalline compound of claim 10.

27-29. (canceled)

30. A method of improving the cognitive performance of a patient, which comprises administering to the patient an effective amount of the crystalline compound of claim 10.

31. (canceled)

32. The method of claim 30, wherein the cognitive performance is rapidity of learning, comprehension, reasoning, or memory.

33. A method of treating, managing or preventing a disease or disorder in a patient, which comprises administering to the patient an effective amount of the crystalline compound of claim 10.

34. (canceled)

35. The method of claim 33, wherein the disease or disorder is Alzheimer's disease, autism, a cognitive disorder, dementia, a learning disorder, or memory loss.

36. The method of claim 35, wherein the learning disorder is dyslexia, dyscalculia, dysgraphia, dysphasia, or dysnomia.

37. A method of preparing a dosage form, which comprises combining crystalline (3′-chlorobiphenyl-4-yl)(1-(pyrimidin-2-yl)piperidin-4-yl)methanone with a pharmaceutically acceptable excipient.

38. The method of claim 37, wherein the dosage form is a liquid dosage form.
